SEQUENCE LISTING (4 sequences)

SEQ ID NO: 1 - DNA, 26 bp, Artificial Sequence
  DNA sequence containing SunI restriction site
agctcgtacgaagcttggtaccggtg

SEQ ID NO: 2 - DNA, 26 bp, Artificial Sequence
  DNA sequence containing SunI restriction site
aattcaccggtaccaagcttcgtacg

SEQ ID NO: 3 - DNA, 20 bp, Artificial Sequence
  PCR primer designed to amplify ampicillin gene from pIPspAdApt10 plasmid
ccgaaaagtgccacctgacg

SEQ ID NO: 4 - DNA, 23 bp, Artificial Sequence
  PCR primer designed to amplify ampicillin gene from pIPspAdApt10 plasmid
taaaaggatctaggtgaagatcc

(19) United States
(12) Patent Application Publication
(10) Pub. No.: US 2002/0177229 A1
(43) Pub. Date: Nov. 28, 2002

(54) Swap/counter selection: a rapid cloning method

(76) Inventors: Ivo Piest, Vinkeveen (NL); Godefridus A.M. Michiels, Leiderdorp (NL); Helmuth H.G. van Es, Hoofddorp (NL); Onno van de Stolpe, Leiden (NL)

Correspondence Address: SYNNESTVEDT & LECHNER, LLP, 2600 ARAMARK TOWER, 1101 MARKET STREET, PHILADELPHIA, PA, 191072950

(21) Appl. No.: 10/051,481

(22) Filed: Jan. 18, 2002

Related U.S. Application Data
(60) Provisional application No. 60/264,757, filed on Jan. 29, 2001.

(30) Foreign Application Priority Data
Jan. 19, 2001 (EP) 01870011.2

(51) Int. Cl. C12P 19/34; C12N 15/74; C12N 15/87

(52) U.S. Cl. 435/455; 435/471; 435/91.2; 435/320.1

(57) ABSTRACT

A method for transferring a polynucleic acid sequence from a donor vector to an acceptor vector wherein said donor vector includes a first antibiotic resistance functioning sequence (ARFS) and said acceptor vector includes a second ARFS comprising:

(a) digesting said donor and acceptor vector with restriction endonucleases, such that said polynucleic acid and restricted donor vector are capable of ligation,

(b) combining the unpurified digestion products into a ligation reaction mixture,

(c) transforming host cells with said mixture,

(d) introducing cells of step (c) onto plates containing a second antibiotic to which cells containing said second ARFS are resistant,

(e) growing colonies of said cells in the presence of a compound that changes color in the presence of the expression product of said first ARFS, and

(f) collecting cells including said polynucleic acid contained in said acceptor vector from colonies that grow and that do not exhibit a color change.

### CROSS REFERENCE TO RELATED APPLICATIONS

This application claims priority to European Patent Application No. 01870011.2, filed Jan. 19, 2001 and U.S. Provisional Application No. 60/264,757, filed Jan. 29, 2001.

### FIELD OF THE INVENTION

The present invention relates to the field of recombinant DNA technology and a procedure for transferring individual polynucleic acid sequences from pre-existing collections of donor vectors into desired acceptor vectors, without the need to isolate the polynucleotide sequence from the donor vector, thereby allowing the procedure to become amenable to automation and miniaturization. In particular, the invention relates to the use of different selection markers on the donor and the acceptor vector combined with a counter selection method to ensure the acceptor vector containing the transferred polynucleic acid sequence has been obtained.

The field of functional genomics relates to high-throughput analysis of gene function by screening large collections of cDNAs in phenotypic assays in order to ascribe potential functions to genes (functional screening). Usually, cDNA libraries consisting of ‘random’ collections of partial and full-length cDNA clones are used in functional screenings. However, recent initiatives have resulted in the availability of large collections of individual sequence-validated full-length cDNA clones, which provide powerful new reagents for functional screenings.

Unfortunately, the re-cloning of large collections of full-length cDNAs in acceptor vectors is currently impossible in an automated setting, since cDNA clones have to be digested with restriction endonucleases and the inserts have to be purified from the donor vector prior to ligation into the desired acceptor vector. The most laborious part of this procedure is the gel-isolation of the insert cDNAs to separate them from the donor vector, and the subsequent purification of the cDNAs from agarose (Sambrook et al. Molecular Cloning: A Laboratory Manual. Cold Spring Harbor Laboratory Press 1989). Although several methods exist for the purification of DNA from agarose or polyacrylamide (PAA), such as phenol extraction, electroelution and purification over glass (beads), none of these methods are compatible with a high-throughput, automated system. Moreover, the loading of the samples on a gel, and the cutting of the desired fragments out of the gel, remains a tedious task that is not easily amenable to automation.

Reported Developments

Recently, two methods were described that circumvent the gel-isolation procedure for recloning (cDNA) inserts, Gateway™ from Life Technologies (U.S. Pat. No. 5,888,732) and Echo™ Cloning System from Invitrogen (U.S. Pat. No. 5,851,808). Both methods make use of unique sequences that surround the cDNA inserts, which sequences are recognized by specific enzymes, namely Integrase for the Gateway™ system and Cre-recombinase for the Echo™ Cloning System. These enzymes are used to swap the inserts from a donor vector to a compatible acceptor vector. However, as these systems were introduced recently, existing large collections of cDNAs are not compatible with these aforementioned cloning systems.

Others (Kirschman J. A. and Cramer J. H. (1988) Gene, 68, 163-165) have described a method whereby a given DNA sequence present in a donor vector can be subcloned into an acceptor vector without purifying the DNA sequence from the donor vector. However, this methodology does not eliminate false-positives wherein the donor vector has been cloned into the acceptor vector resulting in an acceptor vector possessing the drug resistance markers of both the donor and acceptor vectors.

Similarly, Ferguson et al. (Ferguson J. et al. (1981) Gene, 16, 191-197) describe a method of subcloning truncated yeast DNA inserts from an ampicillin resistance vector into a kanamycin resistance vector without purification of the yeast DNA inserts. However, this method also may result in false-positives.

The object of the present invention is to provide a rapid re-cloning method for polynucleic acids to circumvent the problems concerning non-compatibility of the current DNA collections with specific cloning systems.

Further, it is an object of the present invention to provide preferable cloning vectors particularly designed to be used in a rapid re-cloning method.

The present invention is directed to methods whereby a counter selection step identifies false-positives so that only the desired acceptor vectors containing the transferred polynucleic acid sequence can be isolated.

### SUMMARY OF INVENTION

The present invention provides an alternative method for transferring polynucleic acid sequences without purification and makes use of a color-based counter selection step.

The present invention relates to a method for transferring a polynucleic acid sequence from a donor vector to an acceptor vector wherein said donor vector includes a first antibiotic resistance functioning sequence and said acceptor vector includes a second antibiotic resistance functioning sequence comprising:

(a) digesting said donor vector and said acceptor vector with restriction endonucleases, which digesting releases said polynucleic acid from said donor vector and restricts said acceptor vector such that said released polynucleic acid and said restricted donor vector are capable of ligation,

(b) combining the unpurified digestion products including said released polynucleic acid and said restricted acceptor vector into a ligation reaction mixture,

(c) transforming host cells with said mixture of step (b),

(d) introducing said host cells of step (c) onto plates consisting of growth medium containing a second antibiotic to which hosts cells containing said second antibiotic resistance functioning sequence are resistant,

(e) growing distinct colonies of said host cells in the presence of a compound that changes color in the presence of the expression product of said first antibiotic resistance functioning sequence, and

(f) collecting host cells including said polynucleic acid contained in said acceptor vector from colonies that grow on said plates from step (e) and that do not exhibit a color change indicating the presence of said first antibiotic resistance functioning sequence.

The present invention relates further to a method for transferring a first library of unique polynucleic acid sequences included in a library of donor vectors into a second library including each of said unique polynucleic acid sequences in the form of an acceptor vector, wherein said donor vectors include a first antibiotic resistance functioning sequence and said acceptor vectors include a second antibiotic resistance functioning sequence, comprising:

(a) digesting each of said donor vectors of said first library and said acceptor vectors with restriction endonucleases, which digesting releases each of said polynucleic acids from said donor vectors and restricts said acceptor vector such that each of said released polynucleic acids and said restricted donor vector are capable of ligation,

(b) combining into distinct ligation reaction mixture compartments of a third library, each of the unpurified digestion product including each of said released polynucleic acids of said library and said restricted acceptor vector,

(c) transferring each of said distinct ligation reaction mixtures into each of a multiplicity of distinct transformation compartments containing host cells and growth medium containing said second antibiotic and transforming said host cells,

(d) growing distinct colonies of said host cells in each of said compartments in the presence of a compound that changes color in the presence of the expression product of said first antibiotic resistance functioning sequence, and

(e) collecting host cells including said polynucleic acids contained in said acceptor vectors from colonies that grow in said compartments and that do not exhibit a color change indicating the presence of said first antibiotic resistance functioning sequence.

The present invention is more fully described in reference to the accompanying figures and detail description below.

### BRIEF DESCRIPTION OF THE DRAWINGS

FIG. 1: Schematic representation of pIPspAdApt8.

FIG. 2: Schematic representation of pIPspAdApt10.

FIG. 3: Schematic representation of adenoviral acceptor vectors pIPspAdApt8/Zeo (A) and pIPspAdApt10/Zeo (B).

FIG. 4: Schematic representation of pIPspAdApt6-lacZ.

FIG. 5: Schematic representation of universal adenoviral acceptor vector pIPspAdApt10/Zeo-lacZpart.

FIG. 6: Schematic representation of pIPspAdpt6-EGFP.

FIG. 7: Schematic representation of pCLIP-Pac1-EF1α.

FIG. 8: Schematic representation of the swapping/counter selection cloning system. Both the donor vector and the (adenoviral) acceptor vector are digested with compatible restriction endonucleases, inactivated, and reaction mixtures are directly used for the ligation step from which an aliquot is transformed into E. coli. Transformants are selected on antibiotics for which the acceptor vector confers resistance (Y). Transformants are then counter selected by treatment with a chromogenic substrate for the gene product encoded by the donor vector antibiotic resistance functioning sequence.

### DETAILED DESCRIPTION OF THE INVENTION

The present invention is based on a procedure that is easily adapted to automation in a high-throughput setting, which takes advantage of the multiple resistance markers that exist and that can be used to select for a specific vector. In this scenario, acceptor vectors are constructed which contain distinct resistance markers that differ from the resistance marker of the donor vector. After swapping of the polynucleic acid sequence insert from the donor vector to the acceptor vector, the acceptor vector is specifically selected using the unique resistance marker of the acceptor vector followed by a counter selection method that selects against any vector carrying the resistance marker of the donor vector.

In this method, the donor vector is digested with restriction endonucleases that free the polynucleic acid sequence insert from the donor vector backbone. This mixture is directly added to the linearized acceptor vector, without isolation or purification of the polynucleic acid sequence insert. The acceptor vector is linearized with compatible restriction endonucleases and contains a different resistance marker. Following ligation, the mixture is transformed into a suitable host and plated on selection plates containing the antibiotic for which the acceptor vector provides resistance. A counter selection is performed by growing the host cells in the presence of a compound that changes color in the presence of the resistance marker of the donor vector. Cells which grow on the plates and that do not exhibit a color change will not contain donor vector backbone sequences. In this way, only clones containing the acceptor vector will be selected.

The whole procedure can be done in a multiwell format, i.e. on 6, 24, 96 or 384 and even 1536 well plates, and is easy to automate. Most of the procedure can be done in succession on the same plate, simply by adding the next reagents and, therefore, does not require any sophisticated robotics.

In one embodiment the compound is provided in said growth media.

In another embodiment the compound is provided by introducing said compound onto the surface of said plate.

In yet another embodiment, the growth medium comprises said compound and a charged polymer gelling agent that is capable of retarding the diffusion throughout the growth medium of said compound and the product of the interaction of said compound with the expression product of the first antibiotic resistance functioning sequence.

In certain embodiments the charged polymer gelling agent is a polycationic polymer.

In other embodiments the charged polymer gelling agent is a polyanionic polymer.

In certain embodiments the polycationic polymer is chitosan.

In one embodiment the compound includes a negative charge.

In another embodiment the compound includes a positive charge.

In preferred embodiments the compound is a chromogenic beta lactamase substrate.

In a preferred embodiment the compound is nitrocefin.

In preferred embodiments the acceptor vector is capable of homologous recombination with nucleic acid sequences encoding adenoviral genes to form a replication incompetent adenoviral vector.

In a preferred embodiment said donor vector contains an antibiotic resistance functioning sequence selected from the group consisting of β-lactams, macrolides, aminoglycosides, tetracyclines, polypeptides and polymer classes of antibiotics.

In a preferred embodiment said donor vector contains an antibiotic resistance functioning sequence providing resistance to ampicillin.

In a preferred embodiment said acceptor vector contains an antibiotic resistance functioning sequence providing resistance to zeocin.

In yet another embodiment said restriction endonucleases present in said unpurified digestion products in step (b) are inactivated.

In a preferred embodiment said polynucleic acid sequence is part of a set or library of sample nucleic acids that have been individually cloned into donor vectors.

In a preferred embodiment said method of the invention is part of an automated or high-throughput procedure.

In a preferred embodiment said acceptor vector used in a method of the invention as defined above is an expression vector.

In a more preferred embodiment said acceptor vector is a viral expression vector.

In an even more preferred embodiment said acceptor vector is a retroviral vector, and in a most preferred embodiment, said acceptor vector is an adenoviral vector.

In a preferred embodiment said adenoviral vector is an adenoviral adapter vector which contains the left ITR and part of the E2B region, and in which the E1 region has been exchanged for a mammalian promoter, a polylinker sequence, and a polyadenylation signal.

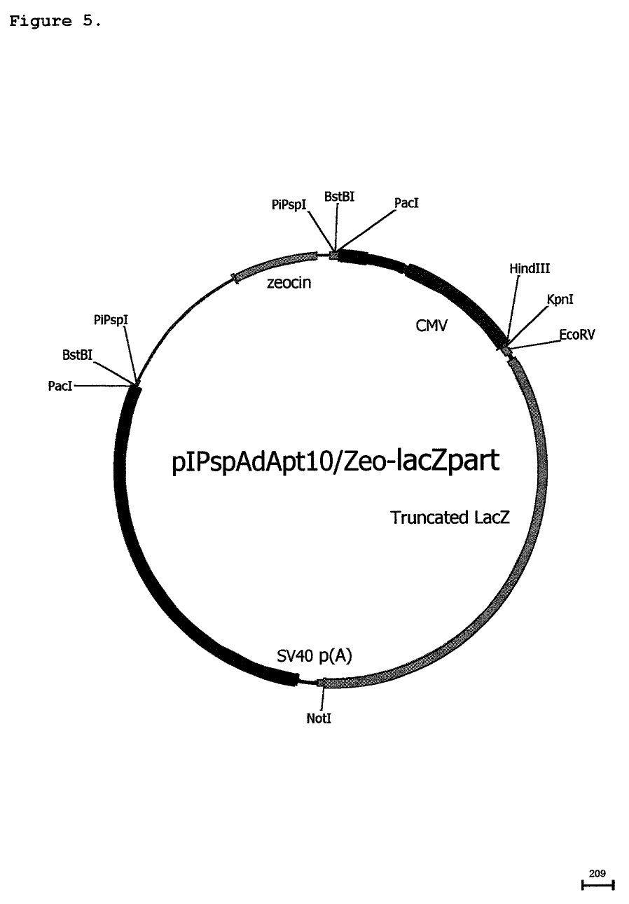

In a more preferred embodiment said adenoviral vector is pIPspAdApt10/Zeo-lacZpart as shown in FIG. 5 which was produced as described in detail in the Examples section.

In a further embodiment the present invention also relates to an adenoviral vector pIPspAdApt10/Zeo-lacZpart as shown in FIG. 5.

Most cloning vectors used today contain the ampicillin resistance gene for selection in Escherichia coli (E. coli). The re-cloning of inserts from one vector to another is routinely performed by digestion of donor and acceptor vectors with compatible restriction endonucleases, isolation of the donor insert and acceptor vector by agarose or polyacrylamide (PAA) gel electrophoresis, followed by purification of the desired DNA fragments from the agarose or PAA gel. The purified DNA fragments are subsequently ligated together, the ligation mixture is transformed into E. coli, and the bacteria are plated on selective, ampicillin-containing medium to select for bacteria that have received a copy of the vector. Subsequently, independent colonies are inoculated and analyzed for the presence of the insert into the acceptor vector. In this regard, the terms “purified” and “purification” relate to the physical separation of the sample nucleic acid from the donor vector, thus digestion or liberation of the sample nucleic acid from the donor vector is not intended.

However, with the use of different selection markers in vectors to be used in a method according to the invention as set out above, it is possible to swap inserts from donor vector to acceptor vector, without the need to purify the polynucleic acid sequence insert from the donor vector. Selection markers that can be used in a method according to the present invention are antibiotic resistance functioning sequences, such as genes encoding antibiotic resistance. The expression product of a given antibiotic resistance functioning sequence will confer resistance to a given antibiotic.

The present invention includes within its scope antibiotic resistance functioning sequences which provide resistance to classes of antibiotics such as the β-lactams, macrolides, aminoglycosides, tetracyclines, polypeptides, polyenes, and nitroimidazole families of antibiotics. Preferred antibiotics include ampicillin-, chloramphenicol-, kanamycin-, zeocin-, penicillin, erythromycin, streptomycin, neomycin, polymyxin B, metronidazole, tinidazole and tetracycline.

A vector containing a specific type of antibiotic resistance functioning sequence can grow in the presence of the respective antibiotic. In the method according to the present invention, a donor vector, conferring resistance to one of the above-mentioned antibiotics, i.e. an antibiotic of the first type, is digested with restriction endonucleases that release the insert from the vector. An acceptor vector, conferring resistance to one of the other above mentioned antibiotics, i.e. an antibiotic of the second type, is digested with compatible restriction endonucleases and added together with the digested donor vector in a ligation reaction mixture. Following transformation of the ligation mixture into a suitable host, such as, for example, bacteria, e.g. E. coli, the host cells are plated on plates containing selective growth medium, i.e. containing one or more antibiotics of the second type. For example, the bacteria are plated on LB-agar plates that contain the antibiotic for which the acceptor vector confers resistance. In this way, only host cells, e.g. bacteria that have been transformed with the acceptor vector can form colonies. The selection of bacteria or other host cells containing an antibiotic resistance marker of the second type can also take place in liquid medium instead of plates. To ensure that only acceptor vector containing the transferred polynucleic acid sequence has been isolated and to eliminate acceptor vector into which the donor sequence has been cloned (false-positives) a counter selection step is performed which identifies by color these false positive colonies.

Reference is made to an antibiotic of a “first type” and an antibiotic of a “second type” to refer to two different types of antibiotic resistance functioning sequences present in the donor and acceptor vectors to be used in a method according to the present invention so that selection and counter selection can take place.

The donor vector to be used in a method according to the present invention can be any vector into which a polynucleic acid sequence has been inserted, including but not limited to vectors containing a bacterial origin of replication and resistance marker, bacterial phage derived vectors such as lambda-, M13-, P1-vectors, and yeast vectors such as yeast artificial chromosomes (YAC).

Polynucleic acid sequences to be sub-cloned in a method according to the present invention are cDNA, genomic DNA, previously cloned DNA, genes, ESTs, synthetic oligonucleotides, randomized sequences, antisense nucleic acids, genetic suppressor elements, ribozymes, mutant zinc fingers, antibody sequences or any combination thereof. In a preferred embodiment, the polynucleic acid is a cDNA encoding a full-length protein, but it might also encode a partial, not full length, protein. In an even more preferred embodiment, the polynucleic acid is part of a set or library of polynucleic acids that are individually cloned into the donor vector, whereby the set or library of polynucleic acids consists of between 2 and 2×10⁷ individual polynucleic acids, and usually between 100 and 1×10⁶ individual polynucleic acids, whereby each individual nucleic acid is contained in a separate compartment, such as individual well in a multi-well plate. In a most preferred embodiment, the complete sequence of the set or library of polynucleic acids is known and can be used to determine the strategy for liberating the inserts comprising the set or library of polynucleic acid sequences from the donor vector.

The acceptor vector to be used in a method according to the present invention can be any vector in which the set or library of polynucleic acids has to be inserted. In a preferred embodiment the acceptor vector is an expression vector, such as, for example, a vector for high level expression in E. coli, a mammalian expression vector, an insect expression vector, a plant expression vector, or a yeast two-hybrid vector. In a more preferred embodiment, the acceptor vector is a viral expression vector, such as, for example, a herpes virus vector, a baculovirus vector, a lentivirus vector, an adeno-associated virus vector, a retrovirus vector, an alphavirus vector, or any combination thereof. Retroviral vectors that can be used include, but are not limited to, Moloney Murine Leukemia Virus (Mo-MuLV)-derived viral vectors such as the pBabe (Morgenstern JP and Land H (1990). Nucleic Acids Res., 18, 3587-3596), deltaMoPyF101 (Havenga MJE et al. (1997) Gene Therapy, 4, 1393-1400) or pMFG vectors. Dranoff G et al. (1993) Proc Natl Acad Sci USA, Apr 15; 90, (8):3539-43). In an even more preferred embodiment, the acceptor vector is an adenoviral vector. In the most preferred embodiment, the acceptor vector is an adenoviral adapter vector (e.g. pclip, pAdApt, pIPspAdApt) which contains the left ITR and part of the E2B region, and in which the E1 region has been exchanged for a mammalian promoter, a polylinker sequence, and a polyadenylation signal. Preferably, at least two acceptor vectors are generated that contain different resistance markers to be compatible with different donor vectors.

Large batches of digested acceptor vector, using all combinations of restriction endonucleases, can be prepared in advance. Batches which show little or no re-ligation in the absence of donor vectors should be used preferentially for the swap/counter selection procedure. However, it is contemplated that the acceptor vector can be digested simultaneously or after digestion of the donor vector. Hence, the donor vector and the acceptor vector can be digested in the same reaction vessel.

The term “compatible restriction endonuclease” as used in step (b) of a method according to the invention as defined above refers to restriction endonucleases (type II) that generate compatible cohesive termini compared to the termini generated in step (a) of the method of the present invention. The restriction endonuclease recognizes specific DNA sequences of dyad symmetry (restriction sites), and cleaves each strand of the substrate DNA on the 5′ end or the 3′ end of the axis of dyad symmetry, resulting in staggered DNA breaks. These breaks yield fragments of DNA that either carry protruding cohesive 5′ or protruding cohesive 3′ termini, respectively. In addition, certain restriction endonucleases cleave both strands of DNA at the axis of dyad symmetry producing blunt ended termini. Hence, compatible cohesive termini are termini that either form hydrogen bonded base pairs (protruding cohesive 5′ and protruding cohesive 3′ termini) if they are incubated under conditions that favor formation of base pairs, or are blunt ended termini, which can be joined together with ligase, such as, for example T4 DNA ligase. It will be understood that cleaved restriction sites can be partially filled in with the deoxyribonucleotide of choice by DNA polymerase to generate compatible termini. It will be understood by the person skilled in the art that the term “compatible restriction endonuclease” also relates to isoschizomers. Digestion is the cleavage of the double stranded substrate DNA by a restriction endonuclease.

The polynucleic acid can be liberated from the donor vector using restriction endonucleases that cut on both sites of the polynucleic acid. These restriction endonucleases may have additional sites within the vector, but preferably not within the polynucleic acid. In general, restriction endonucleases that have been used to generate the donor vector can be used to liberate the polynucleic acid. However, individual members of a set or library of polynucleic acids may have internal sites, e.g. when they have been constructed with the help of so-called adapters. An example of this is the use of EcoRI adapters during the generation of cDNA libraries (Stratagene). In that case, the donor vector must contain sites for other restriction endonucleases between the vector and the polynucleic acid. The sequence of the set or library of polynucleic acids can then be used to group the set or library of polynucleic acids according to the restriction endonucleases that can be used to liberate the individual donor polynucleic acids from the vector.

In the setup of the present invention it is preferred to use those restriction endonucleases that will produce either two different protruding cohesive termini, or one blunt ended terminus and one protruding cohesive terminus, to allow directional cloning as will be clear to those skilled in the art.

Digestion of the donor vector containing the set or library of sample nucleic acids can be performed in microcentrifuge tubes, in a 96 well plate, in a 384 well plate or even in a 1536 well plate. In a preferred scenario, between 0.01 and 0.5 micrograms of donor vector is digested with the appropriate restriction endonucleases for 2 hours at the optimal temperature in a final volume of between 2.5 microliters and 5 microliters. Digestion in multiwell plates can be done in an automated setting by using 96 or 384 channel pipettors such as a Multimek (Beckman) or a Quadra (Tomcat), and multiwell dispensers, for example, a multidrop.

The acceptor vector, which has been digested with appropriate restriction endonucleases to create compatible termini, is added to the digested donor vector in 10× concentrated ligation buffer (e.g. New England Biolabs) such that the final concentration of ligation buffer is 1×. This is done also to inactivate the restriction endonucleases that were used to digest the donor vector and possibly the acceptor vector, since most restriction endonucleases are inactive in lx ligation buffer. Again, this can be done in an automated setting by using 96 or 384 channel pipettors such as a Multimek (Beckman) or a Quadra (Tomcat), and multiwell dispensers, for example, a multidrop. Restriction endonucleases that are still active in lx ligation buffer can be inactivated prior to the addition of the acceptor vector, by incubation at 65° C. for one hour, or by incubation at −20° C. for 16 hours, depending on whether or not they can be heat inactivated, respectively. It will be understood by those skilled in the art that by using isoschizomers to generate the compatible termini, inactivation of the isoschizomers is not necessary per se. The preferred volume for digestion is between 1 and 100 microliters, and more preferably between 5 and 20 microliters.

Following addition of the digested acceptor vector, ligation is carried out. Ligation is the formation of phosphodiester bonds between adjacent 3′-hydroxyl and 5′-phosphate termini in DNA, in this way DNA molecules with compatible termini can be joined. Preferentially, ligation is carried out for 2 hours at room temperature using for instance T4 DNA ligase or any other suitable DNA ligase as will be known to those experienced in the art. Ligation can be done in an automated setting by using 96 or 384 channel pipettors such as a Multimek (Beckman) or a Quadra (Tomcat), and multiwell dispensers, for example, a multidrop. Following ligation, an aliquot of the ligation reaction mixture is used to transform suitable host cells. In particular, an aliquot of the ligation mixture between 0.1 and 10 microliters, or more preferably between 1 and 2 microliters, is taken, added to a new plate and kept on ice. Between 10 and 50 microliters of suitable host cells, such as, for example competent E. coli bacteria (e.g. DH5α, subcloning efficiency; Life Technologies) are added, followed by incubation on ice for 30 minutes, and heat shock at 37° C. for 1 minute. Again, this can be done in an automated setting by using 96 or 384 channel pipettors such as a Multimek (Beckman) or a Quadra (Tomcat), and multiwell dispensers, for example, a multidrop. It will be clear for those skilled in the art, that any suitable host cells as well as any suitable host cell transformation procedure known in the art, amenable to automation, can be used in a method according to the present invention.

After transformation the bacteria are plated on selection media (LB-agar) containing the antibiotic for which the acceptor vector confers resistance, i.e. of the second type, followed by an overnight incubation at 37° C.

As will be clear for those experienced in the art, single colonies are subsequently used to inoculate minipreps in the presence of the antibiotic for which the acceptor vector confers resistance. These small-scale overnight cultures are subsequently used to isolate vector DNA for analysis, such as sequence- or restriction analysis. The present inventors have found that a considerable percentage (up to 50%) of the acceptor vector will contain the donor vector inserted into the acceptor vector, which are false positives. To reduce the amount of false positives in the DNA minipreps, and to reduce the amount of labor, the methods of the present invention utilize a compound that changes color (a “chromogenic compound”) due to its interaction with the expression product of an antibiotic resistance functioning sequence (hereafter “resistance expression product”). The chromogenic compound provides the ability to perform a counter selection step utilizing chromogenic compound.

The chromogenic compound may be any compound that changes color upon interaction with a resistance expression product. The interaction between the chromogenic compound and the resistance expression product is preferably an enzyme-substrate reaction in which the chromogenic compound is a substrate for the resistance expression product. The reaction may take the form of a variety of reactions capable of producing a color change in a chromogenic compound including hydrolysis. As an example the β-lactam class includes ampicillin. The resistance expression product in a donor vector that conferred resistance to ampicillin would be a β-lactamase and an appropriate chromogenic compound would be a β-lactam substrate that changes color due to the enzyme-substitute reaction. In a preferred embodiment utilizing β-lactam class antibiotics, the reaction is an enzyme-substrate reaction involving hydrolysis of β-lactam ring present in a chromogenic compound. As an example, the ampicillin resistance expression product is β-lactamase enzyme that can hydrolyze a β-lactam ring present in a chromogenic compound.

The reaction causes a structural change in the chromogenic compound resulting in a color change. A chromogenic compound in contact with colonies of transfected cells containing acceptor vectors into which the polynucleic acid sequence from the donor vector has been ligated will not change color. In contrast, false positives in which the donor vector itself has been ligated into the acceptor vector will change color due to the reaction between the resistance expression product and the chromogenic compound. This color change may be detected by the human eye or by suitable instruments.

The color of the chromogenic compound after its interaction with the resistance expression product is the basis for identifying false-positives. Bacterial colonies, for example, will come in a variety of colors, depending on the type of bacteria and the ingredients in the growth media provided. In the practice of the present invention, the user will look for a change in the chromogenic compound to the color which indicates the presence of a given resistance expression product. With time, this color may revert back to the original color or all of the colonies may become a given color. Accordingly, the counter selection using the chromogenic compound should be monitored for a suitable period of time for the given chromogenic compound.

The chromogenic compound may have a positive charge or negative charge. Preferred chromogenic compounds include β-lactam ring containing compounds. Examples of suitable chromogenic compounds include nitrocefin, [3-(2,4-Dinitrostyryl)-(6R, 7R)-7-(2-thienylacetamido)-ceph-3-em-4-carboxylic Acid, E-isomer], PADAC [Pyridine-2-azo-p-dimethylanaline cephalosporin], and CENTA (5-thia-1-azabicyclo [4.2.0]oct-2-ene-2-carboxylic acid, 3-[[(3-carboxy-4-nitrophenyl)thio]methyl]-8-oxo-7-[(2-thienylacetyl)amino]-, (6R, 7R)- (9CI); Calbiochen Cat. No. 219475; Jones et al., (1982) J. Clin. Microbiol., 15, 594). In an especially preferred embodiment of the invention, the chromogenic compound is nitrocefin.

The chromogenic compound may be provided in the plates on which the cells are grown, or added to the surface of the plates, or may be added to the cells by introducing the chromogenic compound onto the surface of a plate containing colonies of the cells or directly onto the colonies.

In certain automated embodiments, it may be desirable to use an automated colony picker, which is capable of distinguishing between colonies on the basis of color and picking a colony having a desired color. A variety of colony pickers are commercially available, including automated pickers which utilize a black and white camera that can distinguish between colonies based on the gray level of the colonies. Colony pickers that utilize color cameras may also be used.

A further aspect of the present invention is the use of a charged polymer gelling agent, which is capable of retarding the diffusion of the chromogenic compound and the product of the interaction of the chromogenic compound with the resistance expression product. By retarding diffusion of a chromogenic compound that has changed color, the charged polymer gelling agent stops the chromogenic compound from diffusing to nearby colonies that do not express the resistance expression product and thereby prevents false positives from being selected.

The charged polymer gelling agent may be any suitable charged polymer gelling agent known in the art. The term “gelling agent” as used herein means any agent that is able to form a three-dimensional polymeric network to increase the viscosity of the media to which the gelling agent is added.

The charged polymer gelling agent may be a polycationic polymer or polyanionic polymer. “Polyionic polymers” are polymers containing multiple charged subunits (subunits containing at least 1 negative or positive charge at a selected pH between about 4-10), and having a net negative (polyanionic) or net positive (polycationic) charge at the selected pH.

“Polyanionic polymers” are polyionic polymers in which the charged subunits are ionizable, negatively charged subunits, typically sulfate, sulfonate, or carboxylate, or phosphate groups. Such polyanionic polymers are also referred to herein as “sulfated, sulfonated, carboxylated, or phosphated” polymers, respectively. Exemplary polymers include salts of carboxyalkylcellulose, and alginic acid.

Preferred polyanionic polymers include sulfated proteoglycans, e.g., sulfated heparin, and other sulfated polysaccharides, such as sulfated cellulose or cellulose derivatives, carrageenin and dextran sulfate, mucin, sulfated polypeptides, such as polylysine with sulfated amine groups, and glycopeptides with sulfonate-derivatized saccharide or peptide subunits, and hyaluronic acid.

One type of preferred polyanionic polymer includes sulfated, sulfonated, carboxylated, or phosphated comb-polymer glycoproteins. The polymer generally includes a polymeric backbone, such as a polypeptide, such as one having repeating subunits, such as repeating amino acid subunits. Attached to the backbone, at attachment points spaced along the backbone, are a plurality of polysaccharide side chains. The side chains carry negatively charged sulfate groups, typically several per chain, but an average of at least about 1 negatively charged group per chain.

“Polycationic polymers” are polyionic polymers in which the charged subunits are ionizable, positively charged subunits, typically primary, secondary, or tertiary amine groups, or in which the charged subunits contain quaternary amine groups.

Other polycationic polymers, including other polypeptides with multiple lysine groups, or polyamine polymers, including polyquaternary amines, are also suitable. The sizes of the polyvalent species is preferably less than about 5-10 kilodaltons, and preferably has no more than about 5-10 charged groups/molecule.

Biocompatible water-soluble polycationic polymers include, for example, any polymer having protonated heterocycles attached as pendant groups. As used herein, “water soluble” means that the entire polymer, poly(B)y, must be soluble in aqueous solutions, such as buffered saline or buffered saline with small amounts of added organic solvents as cosolvents, at a temperature between 20° C. and 37° C. In some embodiments, poly(B) will not be sufficiently soluble in aqueous solutions per se but can be brought into solution by grafting with water-soluble poly(A) chains. Examples include polyamines having amine groups on either the polymer backbone or the polymer sidechains, such as poly-L-lysine and other positively charged polyamino acids of natural or synthetic amino acids or mixtures of amino acids, including poly(D-lysine), poly(ornithine), poly(arginine), and poly(histidine), and nonpeptide polyamines such as poly(aminostyrene), poly(aminoacrylate), poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylamidopropyltrimethyl ammonium chloride), and natural or synthetic polysaccharides such as chitosan.

“Polyionic hydrophilic polymers” are polyionic polymers which are soluble in an aqueous solution, at a selected pH between about 3-10, preferably having a partition coefficient, expressed as log n-octanol/water, of less than zero.

In methods of the present invention which utilize a chromogenic compound that has a negative charge, polycationic polymers are preferred. In methods of the present invention which utilize a chromogenic compound that has a positive charge, polyanionic polymers are preferred. In a preferred embodiment, the chromogenic compound nitrocefin is used with the cationic polymer gelling agent chitosan.

The retardation of diffusion of the chromogenic compound and its reaction product is facilitated by a charge-center or electrostatic interaction between the chromogenic compound and the polymer in which the relatively small, charged chromogenic compound is attracted to the larger, oppositely-charged polymer, resulting in a retardation in the diffusion of the chromogenic compound.

The method of the present invention is adaptable to miniaturization and automation, allowing high-throughput conversion of large sets of donor inserts into adenoviral acceptor vectors. Large sets of donor inserts may comprise between 2 and 2×10⁷ different sample nucleic acids, and usually will contain between 100 and 1×10⁶ different polynucleic acids. Miniaturization and automation allows conversion of these large sets of sample nucleic acids at a rate of between 10 and 1×10⁴ different sample nucleic acids per day, and usually between 96 and 6144 different sample nucleic acids per day, and is therefore defined as high-throughput conversion.

The availability of two types of acceptor vectors that differ in their polylinker orientation such as pIPspAdApt8/Zeo and pIPspAdApt10/Zeo, which are described in the examples, will allow the rapid cloning of a set or library of polynucleic acids in the sense and anti-sense orientation. Anti-sense expression has proven to be a valuable tool for knock down strategies.

A set or library of polynucleic acids that were cloned into expression vectors according to the method described in the present invention, can be used to generate transducing vectors. For example, active recombinant viruses can be generated by introducing viral vectors—containing the polynucleic acid—in their respective packaging cell lines such as retroviral vectors in BOSC23 cells (Pear W S et al. (1993), Proc. Natl. Acad. Sci., 90, 8392-8396), or Phoenix cells (Grignani F (1998), Cancer Res., Jan 1;58(l):14-9) and adenovirus vectors (WO99/64582) in for example the PerC6 packaging cell line (US5,994,128). Sets or libraries of the abovementioned transducable vectors can be generated in a high-throughput fashion and used in high-throughput functional screening methods, basically as described in WO99/64582. Chimeric adenovirus vectors (WO00/03029) or adeno-associated virus vectors (WO99/32647) can also be used for such high-throughput screening procedures.

The following examples and figure merely serve to illustrate the present invention and are in no way to be construed as limiting the present invention.

### EXAMPLES

### Example 1

### Construction of Adenoviral Acceptor Vectors

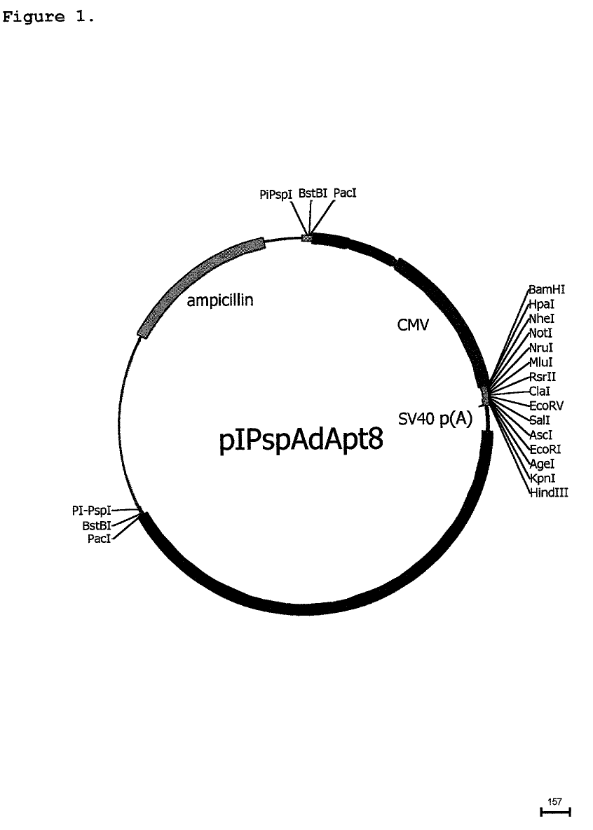

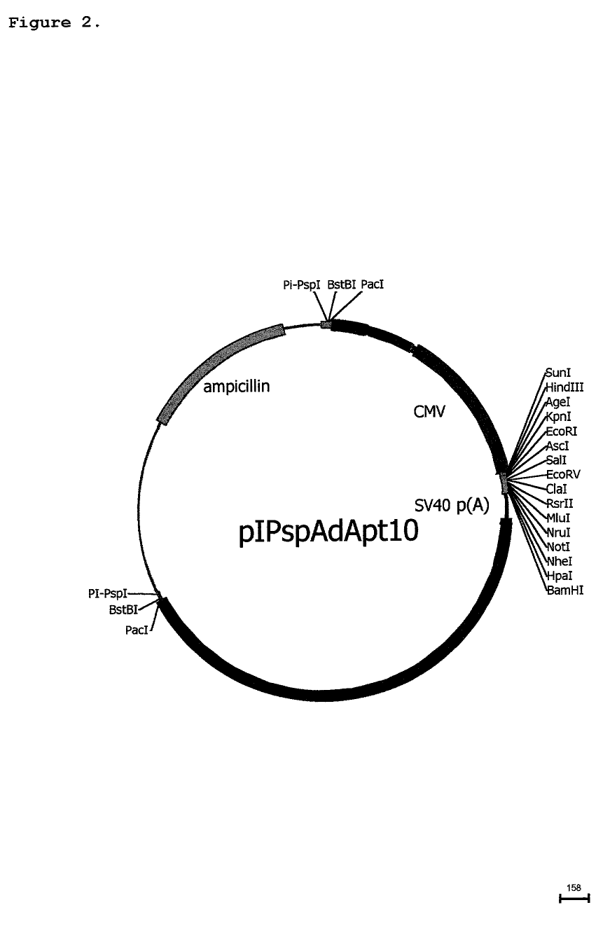

The basis of the present invention is the use of adenoviral acceptor vectors that confer resistance to different antibiotics after transformation to a suitable host cell, such as, for example, E. coli. We have previously described the generation of the adenoviral adapter vectors pIPspAdApt (1-7), which all confer ampicillin resistance after transformation to E. coli (WO 99/64582). Several additions are made to these adapter plasmids. pIPspAdApt8 (FIG. 1) is constructed by digestion of pIPspAdApt6 with HindIII and BamHI. The liberated polylinker is isolated over gel, purified by a Qiaquick gel extraction kit (Qiagen), and cloned into HindIII-BamHI-digested pIPspAdApt4. In this way, the orientation of the polylinker is inverted compared to pIPspAdApt6. To generate pIPspAdApt10, pIPspAdApt6 is digested with HindIII and EcoRI. The oligo's SUNIFOR: 5′-AGCTCGTACGAAGCTTGGTACCGGTG-3′ (SEQ ID NO 1) and SUNIREV: 5′-AATTCACCGGTACCAAGCTTCGTACG-3′ (SEQ ID NO 2) are annealed by heating for 1 minute at 96° C. and cooling overnight to room temperature, followed by ligation into HindIII-EcoRI-digested pIPspAdApt6. In this way, the original HindIII site of pIPspAdApt6 is destroyed, and a SunI site followed by a new HindIII site and the remaining of the pIPspAdApt6 polylinker are inserted, leading to pIPspAdApt10 (FIG. 2).

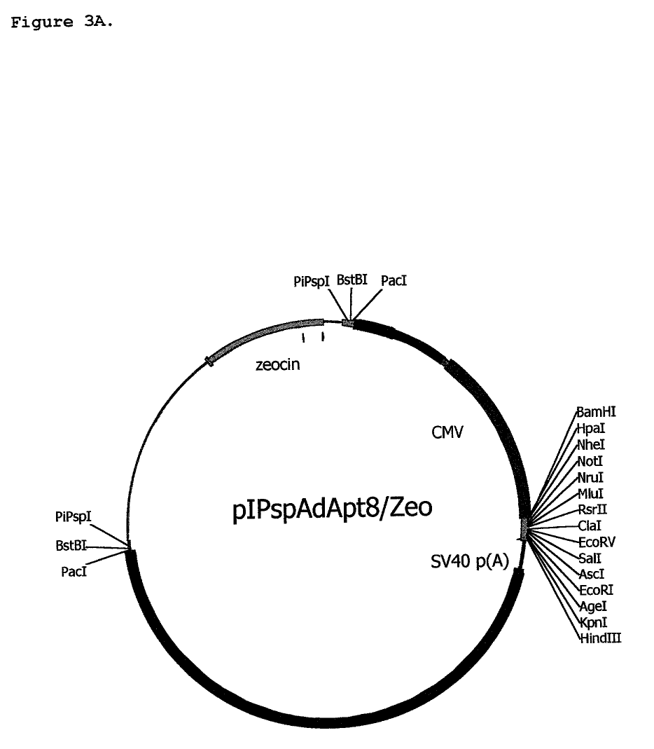

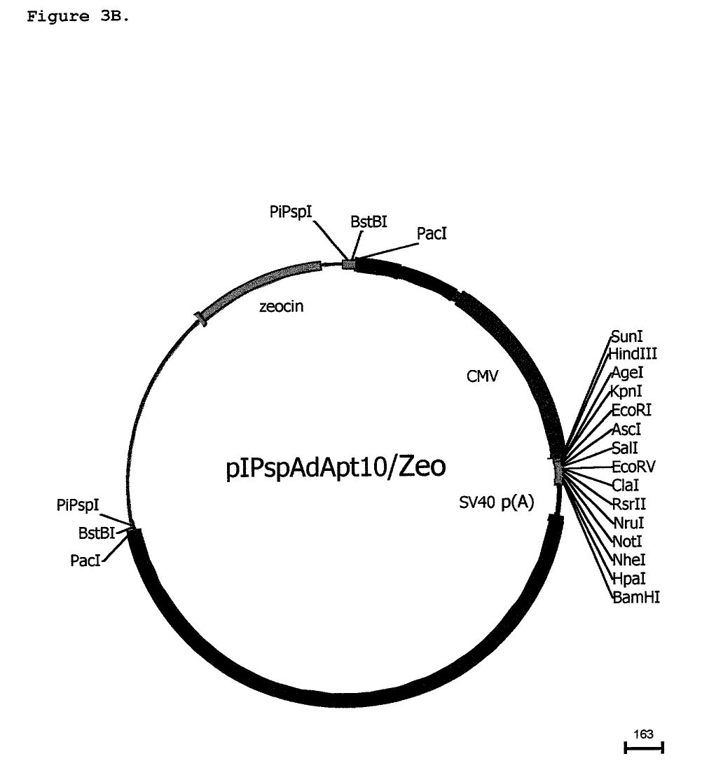

The adenoviral sequences surrounding the ampicillin resistance gene and the bacterial origin of replication of pIPspAdApt6 are amplified by PCR using Elongase (Life Technologies) and primers SspI-forward (CCGAAAAGTGCCACCTGACG, SEQ ID NO 3) and DraI-reverse (TAAAAGGATCTAGGTGAAGATCC, SEQ ID NO 4). The PCR fragment is subsequently treated with T4 polymerase (Life Technologies) to blunt the ends of the fragment. A restriction fragment containing the Sh ble gene is isolated from pZeoSV2+ (InVitrogen) by digestion with KspI and MaeI, followed by treatment with T4 polymerase to blunt the ends of the fragment. Subsequently, the PCR fragment is ligated to the fragment containing the Sh ble gene and, after transformation in E. coli, zeocin-resistant colonies are selected on LB-Zeocin plates. Correct clones are identified by restriction analyses of miniprep DNA. As most of the resulting vector is derived from an in vitro amplified fragment, and therefore might contain errors, we want to ensure that especially the adenoviral part of the vector contained the correct sequences. Therefore, one of the correct DNAs, isolated by the miniprep procedure (miniprep DNA) is digested with PacI, and the resulting 2.2 kb fragment, containing the bacterial origin of replication and the Sh ble gene, is ligated to the 4.1 kb PacI-PacI fragments of pIPspAdApt8 and pIPspAdApt10. The latter fragments contain the adenoviral part of the adapter vectors but differ in their polylinker sequences. Following transformation into E. coli and selection of zeocin-resistant colonies, correct clones are identified by restriction analyses of miniprep DNA. The final vectors are designated pIPspAdApt8/Zeo and pIPspAdApt10/Zeo (FIGS. 3A+B). The sequences surrounding the Sh ble gene are verified by sequence analyses.

### Example 2

### Generation of Universal Adenoviral Acceptor Vector

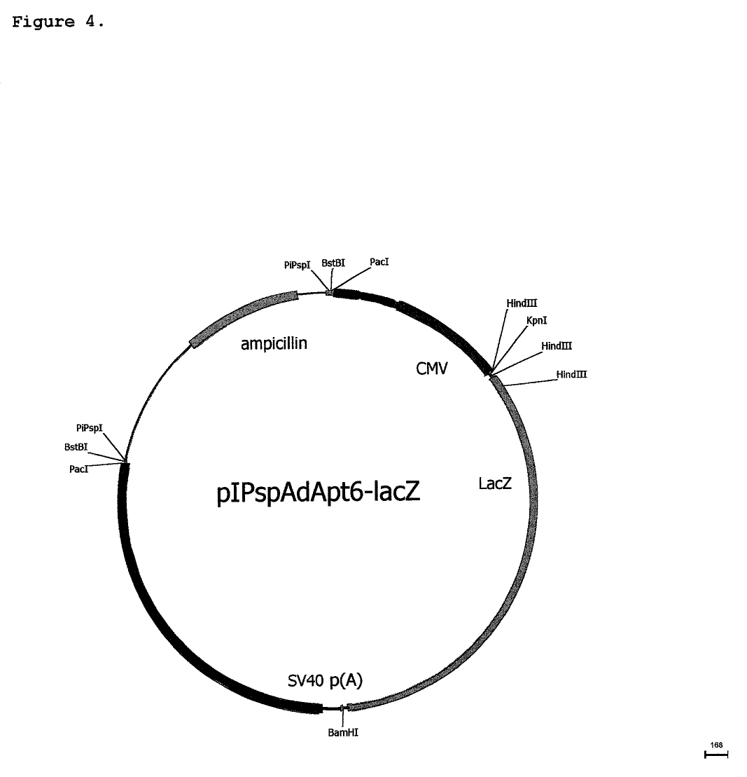

To lower the background of re-ligated acceptor vector due to incomplete digestion of the acceptor vector, a large part of the lacZ gene is inserted into the NruI site of pIPspAdApt10/Zeo (FIG. 3B) to create a universal adenoviral acceptor vector (FIG. 5). The lacZ gene is present in pIPspAdApt6-lacZ (FIG. 4), which is constructed by digestion of pIPspAdApt6 with KpnI and BamHI₁, followed by insertion of the correspondingly digested and purified nls-lacZ gene from pCLIP-lacZ (WO 00/52186). The lacZ gene is isolated by HindIII-EcoRI digestion of pIPspAdApt6-lacZ, blunt-ended by addition of Klenow enzyme (Life Technologies) and deoxyribonucleotides (Amersham-Pharmacia), and ligated into NruI-digested and dephosphorylated pIPspAdApt10/Zeo. Following transformation into E. coli, a correct clone named pIPspAdApt10/Zeo-laczpart (FIG. 5) is selected by restriction analysis.

Using the universal adenoviral acceptor vector, pIPspAdApt10/Zeo-lacZpart, the correctly digested acceptor vector, resulting from digestion with one restriction endonuclease that digests in front of the lacZ gene and one restriction endonuclease that digests behind the lacZ gene, is isolated following separation by agarose gel electrophoresis as it differs in molecular weight from the lacZ insert and the singular digested acceptor vector.

### Example 3

### Feasibility of Swap Procedure

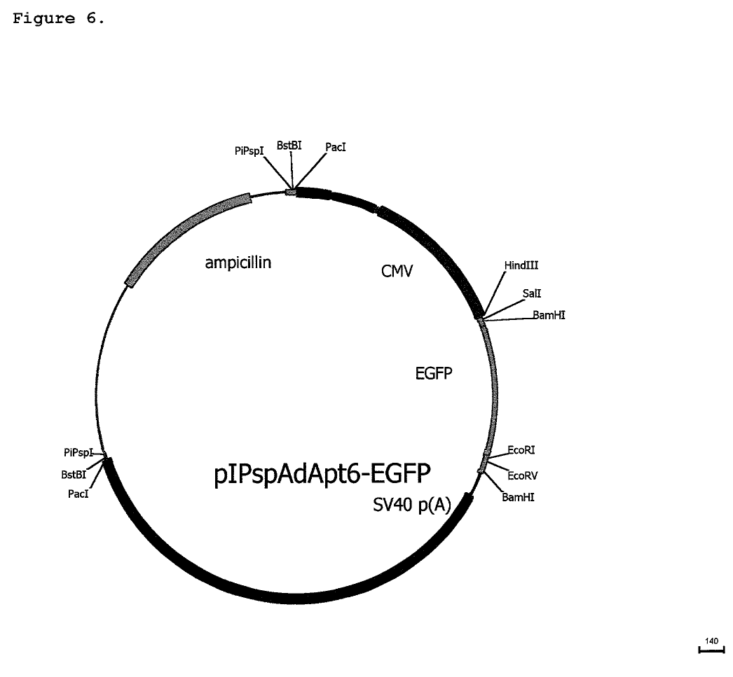

To show the feasibility of the swap procedure, 10 micrograms of the pZeo.SV2+vector (Invitrogen), conferring Zeocin resistance, is digested with the restriction endonucleases EcoRI and HindIII. The vector is gel purified over a 0.8% agarose gel, and DNA is isolated using the QIAquick PCR purification method (pZEO/gel). As an alternative, 10 micrograms of pZeo.SV2+ vector (Invitrogen) is treated for 15 minutes at 37° C. and 15 minutes at 65° C. with Shrimp Alkaline Phosphatase (SAP; Roche) after digestion with EcoRI and HindIII (pZEO/SAP). As donor, pIPspAdApt6-EGFP is used. pIPspAdApt6-EGFP, conferring ampicillin resistance, is constructed by releasing the EGFP insert by HindIII-EcoRI digestion from the plasmid PEGFP (Clontech; catalogue number 6077-1), followed by insertion into HindIII/EcoRI-digested pIPspAdApt6 to generate pIPspAdApt6-EGFP (FIG. 6). 10 micrograms of pIPspAdApt6-EGFP is digested with EcoRI and HindIII to release the EGFP insert from the vector. Half of the digest is loaded on an agarose gel and the EGFP insert is purified using QIAquick PCR purification method (Qiagen). 2.5 micrograms of the remaining 5 micrograms is treated with Shrimp Alkaline Phosphatase (SAP; Roche) for 15 minutes at 37° C. and 15 minutes at 65° C. The remaining 2.5 micrograms are not treated further. Aliquots of all fragments/digests are run on an agarose gel and stained with Ethidium Bromide to compare the amounts of DNA.

Ligations are set up for 4.5 hours at room temperature in a total volume of 10 microliters using 0.5 Units of T4 DNA ligase in 1× ligation buffer (Life Technologies) to compare the SAP-treated versus the gel-purified pZeo.SV2+ vector. As donors, the HindIII/EcoRI-digested pIPspAdApt6-EGFP (EGFP) is compared to the SAP-treated, HindIII/EcoRI-digested pIPspAdApt6-EGFP (EGFP/SAP), and to the gel-purified HindIII/EcoRI EGFP insert (EGFP/gel). Two molar ratios of pZeo.SV2+ acceptor vector and donor EGFP insert are used in the ligations: 1:1 and 3:1.

1 microliter of all ligation reactions is transformed to MAX efficiency Stbl2 competent cells (Life Technologies), according to the protocol of the manufacturer. Following transformation, cells are plated on a LB-agar-Zeocin plate. 10 colonies are picked from all plates containing the non-purified EGFP as donor and 5 colonies from the plates containing the gel-purified EGFP donor fragment. Miniprep DNA is digested by EcoRI and HindIII to analyze the presence of the 3.5 kb pZeo.SV2+ vector and the 0.7 kb EGFP fragment.

The following results are obtained for the different acceptor (ZEO/SAP and ZEO/gel) and donor (EGFP, EGFP/gel and EGFP/SAP) combinations:

Ratio 1:1 (pZEO/SAP:EGFP)

8 out of 10 colonies show the correct restriction pattern

Ratio 3:1 (pZEO/SAP:EGFP)

4 out of 10 colonies show the correct restriction pattern

Ratio 1:1 (pZEO/SAP:EGFP/gel)

5 out of 5 colonies show the correct restriction pattern

Ratio 3:1 (pZEO/SAP:EGFP/gel)

5 out of 5 colonies show the correct restriction pattern

Ratio 1:1 (pZEO/gel:EGFP/SAP)

9 out of 10 colonies show the correct restriction pattern

Ratio 3:1 (pZEO/gel:EGFP/SAP)

8 out of 10 colonies show the correct restriction pattern

Ratio 1:1 (pZEO/gel:EGFP/gel)

5 out of 5 colonies show the correct restriction pattern

Ratio 3:1 (pZEO/gel:EGFP/gel)

5 out of 5 colonies show the correct restriction pattern

Conclusion: The swap cloning procedure works. In this set up, a molar excess of SAP-treated acceptor vector is not preferred.

To further optimize the swapping efficiency, the ratios of gel-purified HindIII/EcoRI-digested Zeo.SV2+ acceptor (pZEO/gel) and SAP-treated HindIII/EcoRI-digested PIPspAdApt6-EGFP donor (EGFP/SAP) are further modified. The fragments used are identical to those mentioned above.

Ratio 1:2 (pZEO/gel:EGFP/SAP)

10 out of 10 minipreps selected on Zeocin-containing plates and grown in Zeocin-containing LB are correct

Ratio 2:1 (pZEO/gel:EGFP/SAP)

8 out of 10 minipreps selected on Zeocin-containing plates and grown in Zeocin-containing LB are correct

Ratio 5:1 (pZEO/gel:EGFP/SAP)

9 out of 10 minipreps selected on Zeocin-containing plates and grown in Zeocin-containing LB are correct

Conclusion: There is no clear relation between the acceptor:donor ratio and the efficiency of swapping. The overall efficiency of gel-purified acceptor is good.

In a next experiment, the gel-purified, HindIII/EcoRI-digested pZeo.SV2+ acceptor (pZEO/gel) is ligated in different ratios to HindIII/EcoRI-digested pIPspAdApt6-EGFP, which is not treated with SAP (EGFP):

Ratio 1:1 (pZEO/gel:EGFP)

17 out of 20 minipreps selected on Zeocin-containing plates and grown in Zeocin-containing LB are correct

Ratio 1:3 (pZEO/gel:EGFP)

16 out of 20 minipreps selected on Zeocin-containing plates and grown in Zeocin-containing LB are correct

Ratio 3:1 (pZEO/gel:EGFP)

14 out of 20 minipreps selected on Zeocin-containing plates and grown in Zeocin-containing LB are correct

Conclusion: SAP treatment of donor DNA is not required

TABLE 1
Summary of the different donor (EGFP) and
acceptor (ZEO) ligation combinations
(correct clones/tested clones)

| Ratio | | donor: | donor: | donor: |
|---|---|---|---|---|
| acceptor/donor: | acceptor: | EGFP/gel | EGFP/SAP | EGFP |
| 1:1 | pZEO/gel | 5/5 | 9/10 | 17/20 |
| 1:2 | pZEO/gel | | 10/10 | |
| 1:3 | pZEO/gel | | | 16/20 |
| 2:1 | pZEO/gel | | 8/10 | |
| 3:1 | pZEO/gel | | 8/10 | 16/20 |
| 5:1 | pZEO/gel | 5/5 | 9/10 | |
| 1:1 | PZEO/SAP | 5/5 | | 8/10 |
| 3:1 | PZEO/SAP | 5/5 | | 4/10 |

General conclusion: Overall efficiency of gel-purified acceptor is good. SAP treatment of donor DNA is not required. Again, there is no clear relation between acceptor:donor ratio and efficiency of swapping, although a ratio of 1:1 seems optimal.

### Example 4

### Insert Swapping with Adenoviral Acceptor Vector

To show the feasibility of the swapping procedure with an adenoviral acceptor vector, pIPspAdapt10/Zeo-lacZpart is digested with SalI and NotI in React 3 (Life Technologies) and the linearized vector band is isolated over a 0.8% agarose gel and purified using a Gene Clean Spin kit (Bio101). Donor vectors are derived from a human leukocyte cDNA library in pSport, which confers ampicillin resistance (obtained from Life Technologies). Miniprep DNA from 4 different cDNA clones, with insert sizes of about 0.6 kilobases (kb), 1.6 kb, 2 kb, and 3 kb, as judged by estimation of the insert sizes on a 0.8% agarose gel, is also digested with SalI and NotI for 2 hours at 37° C., followed by an overnight storage at −20° C. Prior to ligation, the digests are incubated for 40 minutes at 65° C. to inactivate the restriction endonucleases. An aliquot of digested donor and purified acceptor DNA is run on a 0.8% agarose gel to compare the staining intensities of the donor insert bands and the acceptor vector band by ethidium bromide staining.

Equal molar amounts, as judged from the staining intensities, of donor insert, in the presence of donor vector, and acceptor vector are ligated in 5 microliters containing 1× ligation buffer and 1.0 Unit of T4 DNA ligase (Life Technologies) for 3.5 hours at room temperature. 1 microliter of this ligation mixture is transformed to subcloning efficiency DH5α cells (Life Technologies) according to the protocol of the manufacturer, and cells are plated on an LB plate containing 50 μg/ml zeocin (Invitrogen). All transformations yielded over 150 colonies on the LB-zeocin plates.

10 colonies of each ligation are inoculated in LB-zeocin medium and grown overnight at 37° C. Following miniprep DNA isolation, miniprep DNA is digested with SalI and NotI, and run on a 0.8% agarose gel.

Of the 10 colonies which are derived from the swapping of the 0.6 kb insert, 8 colonies yielded correct pIPspAdapt10/Zeo vectors from which the 0.6 kb insert could be released with digestion by SalI and NotI.

Of the 10 colonies which are derived from the swapping of the 1.6 kb insert, 7 colonies yielded correct pIPspAdapt10/Zeo vectors from which the 1.6 kb insert could be released with digestion by SalI and NotI.

Of the 10 colonies which are derived from the swapping of the 2 kb insert, 8 colonies yielded correct pIPspAdapt10/Zeo vectors from which the 2 kb insert could be released with digestion by SalI and NotI.

Of the 10 colonies which are derived from the swapping of the 3 kb insert, 5 colonies yielded correct pIPspAdapt10/Zeo vectors from which the 3 kb insert could be released with digestion by SalI and NotI.

Thus, the overall efficiency is 28/40=70% of correctly swapped sample nucleic acids.

### Example 5

### Swap/Counter Selection

We note that most of the false positive colonies that grew on LB-zeocin show a similar restriction pattern after miniprep analysis, and suspect that this could be due to ligation of the donor vector in the acceptor vector. To test this, several of the false positive colonies are streaked out on an ampicillin-containing LB-agar and a zeocin-containing LB-agar plate. Most of them indeed are able to grow on both ampicillin plates and zeocin plates. This made it possible to use the ability to grow on ampicillin as a way to counter select for false positives in the swap procedure.

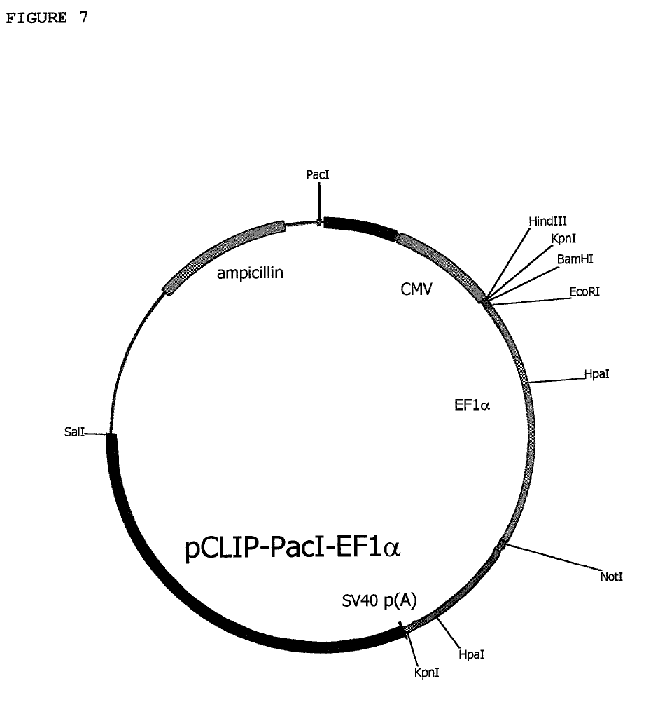

To further verify the use of counter selection, a cDNA encoding human Elongation Factor 1α (EF1α derived from the human leucocyte cDNA library in pSport), is isolated by SalI and NotI digestion, followed by treatment with T4 polymerase (Life Technologies) in the presence of dNTPs to generate blunt ends. The insert is cloned into the adenoviral adapter vector pCLIP-PacI (WO 99/55132) that is digested with EcoRV and treated with Shrimp Alkaline Phosphatase (Life Technologies) to diminish religation of the vector. A correct pCLIP-PacI-EF1α (FIG. 7) as judged by restriction analysis and confirmed by sequence analysis, is digested by MluI and NotI for 2 hours and stored at −20° C.

Two independently generated batches of MluI-NotI digested universal pIPspAdapt10/Zeo-lacZpart acceptor vector (Example 2), termed batch 1 and batch 2, are isolated over an agarose gel and purified using a Gene Clean Spin DNA isolation kit from BIO101.

Ligations are set up in a final volume of 10 microliters containing lx ligation buffer and 10 Units of T4 ligase (New England Biolabs). The amounts of digested pCLIP-PacI-EF1α donor and purified pIPspAdapt10/Zeo-lacZpart acceptor are such that the molar ratio of donor insert to acceptor vector is 1:1, as judged by comparing the staining intensities of the donor insert bands and the acceptor vector band.

Ligations are carried out for 24 hours at 12° C., followed by transformation into subcloning efficiency DH5α competent cells of a 1 microliter aliquot of the ligation mixture. After transformation, cells are plated on zeocin-containing LB-agar plates. 30 colonies of each transformation are subsequently streaked out on both ampicillin- and zeocin-containing LB-agar plates.

Of the 30 colonies derived from acceptor batch 1, 9 colonies grew also on ampicillin-containing plates. Of the 30 colonies derived from acceptor batch 2, 12 colonies grew also on ampicillin-containing plates.

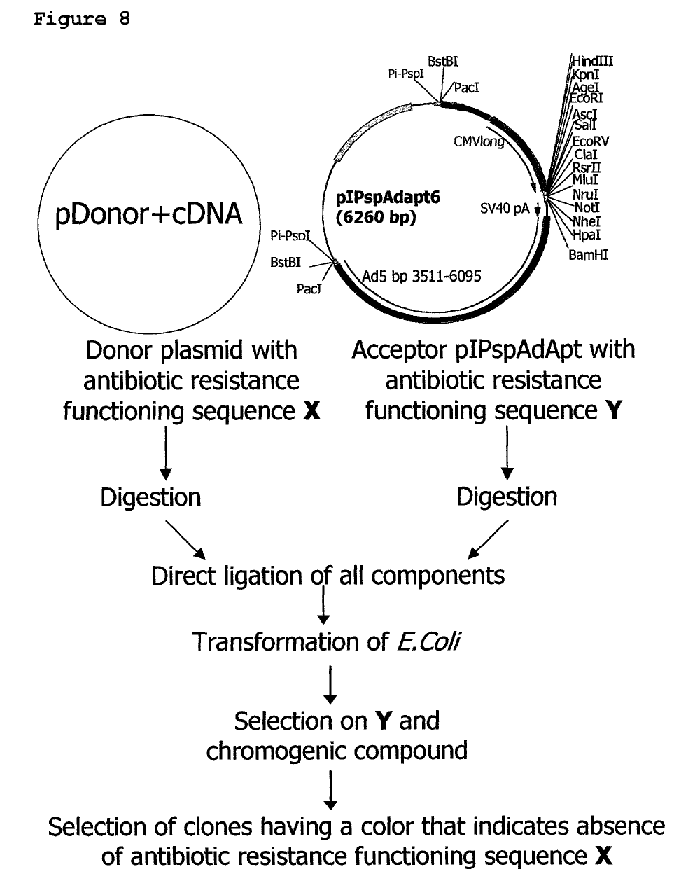

15 minicultures are inoculated from colonies that did not grow on ampicillin plates. Miniprep analysis shows that 100% of the clones derived from batch 1 of the universal pIPspAdapt10/Zeo-lacZpart acceptor vector and 93% of the clones derived from batch 2 of the universal pIPspAdapt10/Zeo-lacZpart acceptor vector contain the correctly swapped EF1α insert. This shows that the swap procedure, in combination with counter selection for the donor vector (FIG. 8), is a very efficient method for the conversion of a cDNA insert from a donor vector to an acceptor vector.

### Example 6

### Swap/Counter Selection Using Chromogenic β-Lactamase Substrates

We note that most of the false positive colonies that grew on LB-zeocin showed a similar restriction pattern after miniprep analysis, and suspect that this could be due to ligation of the donor vector into the acceptor vector. To test this, several of the false positive colonies are streaked out on an ampicillin-containing LB-agar and a zeocin-containing LB-agar plate. Most of them indeed are able to grow on both ampicillin plates and zeocin plates.

To counter select for false positives a chromogenic compound that indicates the presence of the ampicillin resistance gene product is utilized. The ampicillin resistance gene is a β-lactamase that confers ampicillin resistance to bacterial cells by catalyzing cleavage of the -lactam group of ampicillin. In the presence of β-lactamase, a chromogenic β-lactamase substrate will be cleaved and change color.

One such chromogenic β-lactamase substrate is Nitrocefin [3-(2,4-Dinitrostyryl)-(6R, 7R)-7-(2-thienylacetamido)-ceph-3-em-4-carboxylic Acid, E-isomer]. Nitrocefin is converted from yellow (λₘₐₓ at pH 7.0=390 nm) to red (λmax at pH 7.0=486 nm) as the amide bond in the β-lactam ring is hydrolyzed by β-lactamase.

To test a counter selection method utilizing a chromogenic compound, a cDNA encoding human Elongation Factor 1α (EF1α derived from the human leukocyte cDNA library in psport), is isolated by SalI and NotI digestion, followed by treatment with T4 polymerase (Life Technologies) in the presence of dNTP's to generate blunt ends. The insert is cloned into the adenoviral adapter vector pCLIP-PacI (WO 99/55132) that is digested with EcoRV and treated with Shrimp Alkaline Phosphatase (Life Technologies) to diminish re-ligation of the vector. A correct pCLIP-PacI-EF1α(FIG. 7) as judged by restriction analysis and confirmed by sequence analysis, is digested by MluI and NotI for 2 hours and stored at −20° C.

Two independently generated batches of MluI-NotI digested universal pIPspAdapt10/Zeo-lacZpart acceptor vector (Example 2), termed batch 1 and batch 2, are isolated over an agarose gel and purified using a Gene Clean Spin DNA isolation kit from BIO101.

Ligations are performed in a final volume of 10-microliters containing 1× ligation buffer and 10 Units of T4 ligase (New England Biolabs). The amounts of digested pCLIP-PacI-EF1α donor and purified pIPspAdapt10/Zeo-lacZpart acceptor are such that the molar ratio of donor insert to acceptor vector is 1:1, as judged by comparing the staining intensities of the donor insert bands and the acceptor vector band.

Ligations are carried out for 24 hours at 12° C., followed by transformation into DH5α competent cells of a 1-microliter aliquot of the ligation mixture. After transformation, cells are plated on zeocin-containing LB-agar plates.

A nitrocefin solution is prepared according to the manufacturers instructions (Calbiochem®, Catalog No. 484400) in 0.1M phosphate buffer with 5% DMSO. A “direct plate method” in which one drop of the nitrocefin solution is added to the surface of a bacterial colony is used for rapid detection of β-lactamase activity in bacterial colonies.

Several (4 to 5) colonies of one transformation plate are treated with a drop of nitrocefin solution at the same time, so color changes between these colonies can be detected. Within half a minute β-lactamase producers turn red, while those cells lacking β-lactamase activity remain yellow. Both β-lactamase positive and negative colonies are inoculated for mini-prep isolation. The presence of either the pCLIP-PacI or the EF1α insert is confirmed by restriction analysis. All the β-lactamase positive colonies, as detected by nitrocefin, should have the PCLIP-PacI as insert. Conversely, all the β-lactamase negative colonies should have EF1α as insert. Thus, the use of a chromogenic compound to detect the presence of the ampicillin resistance gene product facilitates the screening of plasmids containing the desired insert.

As an alternative to the drop-wise addition of nitrocefin solution to the colonies, the chromogenic β-lactamase substrate can also be incorporated with zeocin into the LB-agar plates. Similarly, the chromogenic β-lactamase substrate solution can also be streaked or sprayed onto the surface of the plates either before or after the colonies have grown.

In order to prevent cleaved chromogenic β-lactamase substrate from diffusing from cells expressing β-lactamase to those colonies which do not express β-lactamase, a solution or gel containing a charged polymer gelling agent such as the cationic polymer gelling agent chitosan can be added to the plates. The charged polymer gelling agent can be added to the medium at the time the LB-agar plates are being poured, after the LB-agar plates have hardened, or after the colonies have grown.

1. A method for transferring a polynucleic acid sequence from a donor vector to an acceptor vector wherein said donor vector includes a first antibiotic resistance functioning sequence and said acceptor vector includes a second antibiotic resistance functioning sequence comprising:
(a) digesting said donor vector and said acceptor vector with restriction endonucleases, which digesting releases said polynucleic acid from said donor vector and restricts said acceptor vector such that said released polynucleic acid and said restricted donor vector are capable of ligation,
(b) combining the unpurified digestion products including said released polynucleic acid and said restricted acceptor vector into a ligation reaction mixture,
(c) transforming host cells with said mixture of step (b),
(d) introducing said host cells of step (c) onto plates consisting of growth medium containing a second antibiotic to which hosts cells containing said second antibiotic resistance functioning sequence are resistant,
(e) growing distinct colonies of said host cells in the presence of a compound that changes color in the presence of the expression product of said first antibiotic resistance functioning sequence, and
(f) collecting host cells including said polynucleic acid contained in said acceptor vector from colonies that grow on said plates from step (e) and that do not exhibit a color change indicating the presence of said first antibiotic resistance functioning sequence.

2. The method of claim 1 wherein said compound is provided in said growth media.

3. The method of claim 1 wherein said compound is provided by introducing said compound onto the surface of said plate.

4. The method of claim 1 wherein said growth medium comprises said compound and a charged polymer gelling agent which is capable of retarding the diffusion of said compound and the product of the interaction of said compound with the expression product of the first antibiotic resistance functioning sequence.

5. The method of claim 4 wherein said charged polymer gelling agent is a polycationic polymer.

6. The method of claim 4 wherein said charged polymer gelling agent is a polyanionic polymer.

7. The method of claim 5 wherein said polycationic polymer is chitosan.

8. The method of claim 5 wherein said compound includes a negative charge.

9. The method of claim 6 wherein said compound includes a positive charge.

10. The method of claim 1 wherein said compound is a chromogenic beta lactamase substrate.

11. The method of claim 10 wherein said compound is nitrocefin.

12. The method of claim 1 wherein said acceptor vector is capable of homologous recombination with nucleic acid sequences encoding adenoviral genes to form a replication incompetent adenoviral vector.

13. The method of claim 1 wherein said first antibiotic resistance functioning sequence provides resistance to an antibiotic selected from the group consisting of β-lactam, macrolide, aminoglycoside, tetracycline, polypeptide, polyene, and nitroimidazole classes of antibiotics.

14. The method of claim 1 wherein said first antibiotic resistance functioning sequence provides resistance to ampicillin.

15. The method of claim 1 wherein said second antibiotic resistance functioning sequence provides resistance to zeocin.

16. The method of claim 1 wherein said restriction endonucleases present in said unpurified digestion products in step (b) are inactivated.

17. A method for transferring a first library of unique polynucleic acid sequences included in a library of donor vectors into a second library including each of said unique polynucleic acid sequences in the form of an acceptor vector, wherein said donor vectors include a first antibiotic resistance functioning sequence and said acceptor vectors include a second antibiotic resistance functioning sequence, comprising:
(a) digesting each of said donor vectors of said first library and said acceptor vectors with restriction endonucleases, which digesting releases each of said polynucleic acids from said donor vectors and restricts said acceptor vector such that each of said released polynucleic acids and said restricted donor vector are capable of ligation,
(b) combining into distinct ligation reaction mixture compartments of a third library, each of the unpurified digestion product including each of said released polynucleic acids of said library and said restricted acceptor vector,
(c) transferring each of said distinct ligation reaction mixtures into each of a multiplicity of distinct transformation compartments containing host cells and growth medium containing said second antibiotic and transforming said host cells,
(d) growing distinct colonies of said host cells in each of said compartments in the presence of a compound that changes color in the presence of the expression product of said first antibiotic resistance functioning sequence, and
(e) collecting host cells including said polynucleic acids contained in said acceptor vectors from colonies that grow in said compartments and that do not exhibit a color change indicating the presence of said first antibiotic resistance functioning sequence.

18. The method of claim 1 wherein said polynucleic acid sequence is part of a set or library of polynucleic acid sequences that are individually cloned into donor vectors.

19. The method of claim 1 wherein said method is part of an automated or high-throughput process.

20. The method of claim 1 wherein said acceptor vector is an expression vector.

21. The method of claim 1 wherein said acceptor vector is a viral expression vector.

22. The method of claim 1 wherein said acceptor vector is a retroviral vector.

23. The method of claim 1 wherein said acceptor vector is an adenoviral vector.

24. The method of claim 23 wherein said adenoviral vector is an adenoviral adapter vector which contains the left ITR and part of the E2B region, and in which the E1 region has been exchanged for a mammalian promoter, a polylinker sequence, and a polyadenylation signal.

25. The method of claim 24 wherein said adenoviral vector is pIPspAdApt10/Zeo-lacZpart as shown in FIG. 5.

26. An adenoviral vector as defined in claim 25.
